(19) United States
(12) Patent Application Publication
(10) Pub. No.: US 2008/0293965 A1
(43) Pub. Date: Nov. 27, 2008

(54) Process for the Dynamic Resolution of (Substituted) (R)- or (S)- Mandelic Acid

(76) Inventor: Bo Lars Gustaf Bosson, Sodertalje (SE)

Correspondence Address: MORGAN LEWIS & BOCKIUS LLP, 1111 PENNSYLVANIA AVENUE NW, WASHINGTON, DC, 20004, US

(21) Appl. No.: 11/913,224

(22) PCT Filed: May 22, 2006
(86) PCT No.: PCT/GB2006/001861

§ 371 Date: Jul. 28, 2008

(30) Foreign Application Priority Data
May 24, 2005 (GB) 0510546.5

(51) Int. Cl. C07C 59/50

(52) U.S. Cl. 562/470

(57) ABSTRACT

The present invention relates to a process for the resolution of mandelic acid derivative enantiomers from (racemic) mixtures by salt formation (see, for example, formula IIa) with chiral base cyclic amides, and racemisation of the unresolved enantiomer in the same process, wherein an additional racemising base may optionally be used, employing an acid:total base (i.e. cyclic amide and optional additional base) molar ratio of at least 1:1; provided that the cyclic amide base is present in a molar ratio of at least 0.75; and to the use of the resolved mandelic acid derivatives as intermediates suitable for large-scale manufacturing of, for example, pharmaceutical compounds; wherein R is selected from CHF₂, H, C₁₋₆ Alkyl, CH₂F, CHCl₂ and CClF₂; and wherein n is 0, 1 or 2; R₁ is H or C₁₋₆ Alkyl and X is H, halo or C₁₋₆ Alkyl.

### FIELD OF THE INVENTION

The present invention relates to a new process for the preparation and resolution of mandelic acid derivatives from (racemic) mandelic acid derivative mixtures, by simultaneous resolution (by salt formation) and racemisation with chiral base cyclic amides. The invention also relates to the use of the resolved mandelic acid derivatives as intermediates suitable for large-scale manufacturing of, for example, pharmaceutical compounds.

### BACKGROUND

Mandelic acids are used in the manufacture of a range of interesting molecules, such as pharmaceuticals. The present invention relates in particular to the preparation and use of resolved mandelic acid derivatives as intermediates suitable for large-scale manufacturing of, for example pharmaceutical compounds, e.g. compounds as described in WO 02/44145.

In PCT application PCT/GB2004/004964 (priority date 28 Nov. 2003) racemic mandelic acid derivatives may be resolved by salt formation with chiral base cyclic amides, such as proline amide. In that application certain metal salts, and certain amine salts of mandelic acid derivatives (particularly (R)-3-chloro,5-difluoro-methoxy mandelic acid) are also described.

In particular, there is disclosed a process for resolving (R)- or (S)-optionally substituted mandelic acids from racemic mixtures of said optionally substituted mandelic acids by salt formation with a chiral base (D)- or (L)-cyclic amide, comprising the steps:

(a) forming a mixture in a solvent, or mixture of solvents, of a racemic, optionally substituted, mandelic acid; and a chiral base (D)- or (L)-cyclic amide, wherein the chiral base used is either (D) for separation of (R)-mandelic acids, or (L) for separation of (S)-mandelic acids, at an acid:base molar ratio of 1:0.25-0.75; and wherein the mixture may optionally contain water in the range of 5 to 15% (vol.) of solvent; and
(b) separating the respective (R)/(D) or (S)/(L) mandelic acid-cyclic amide salt.

It is to be understood that said “(R)- or (S)-optionally substituted mandelic acids” may be as described in WO 02/44145, and wherein said definitions and disclosed optionally substituted mandelic acids are incorporated into this specification by reference.

It is also to be understood that said “(R)- or (S)-substituted mandelic acids” may be those mandelic acid fragments of the molecules described in WO 02/44145, and wherein said definitions and disclosed substituted mandelic acids are incorporated into this specification by reference. Also incorporated into this specification by reference are details and examples of preparation of such substituted mandelic acids described in WO 02/44145 (for example, Example 1 therein).

A general outline of the process in PCT application PCT/GB2004/004964 is as follows (wherein R, R₁, X and n are as defined therein):

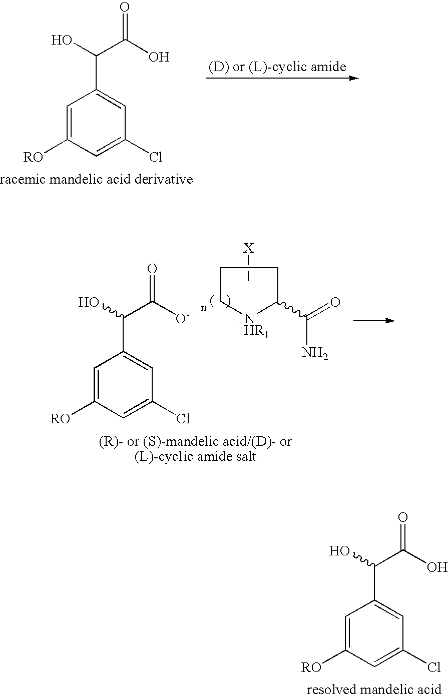

In the above scheme, preferably R₁ and X are both H, and R is —CHF₂.

In PCT application PCT/GB2004/004964, once the desired (“right”) mandelic acid/prolinamide (MAPA) salt has been isolated by filtration, the mother liquors containing an excess of the other (“wrong”) mandelic acid enantiomer (and also some unprecipitated prolinamide salt of the “right” mandelic acid) may be racemised—see racemisation scheme illustrated below. The resulting racemate may again be used in the process of the invention to isolate more of the desired enantiomer. This racemisation/recycling process may be repeated a number of times to obtain higher yields of the desired enantiomer, for example two/three recycles may permit up to 70%-80% overall yield of the “right” mandelic acid.

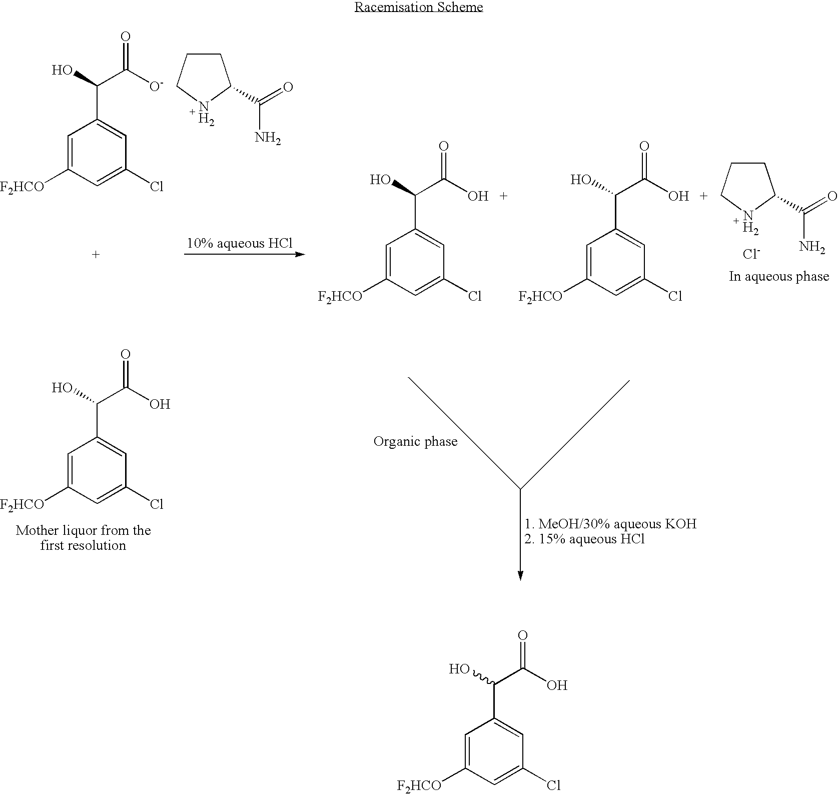

Although the racemisation/recycling process may permit higher yields of the desired enantiomer to be obtained, there remains an ongoing need for further processes which are more efficient (for example, by avoiding repeated work-up and recycle steps) and/or produce even higher yields.

The combination of resolution processes with in situ racemisation to give crystallisation-induced asymmetric transformations has been reported (Ebbers, E. J.; Ariaans, G. J. A.; Bruggink, A.; Zwanenburg, B. Tetrahedron Assymetry 1999, 10, 3701-3718). In particular, crystallisation-induced asymmetric transformations of mandelic acid using alpha-methylbenzylamine in combination with DABCO (1,4-Diazabicyclo[2.2.2]octane), DBN (1,5-Diazabicyclo[4.3.0]non-5-ene) and TBD (1,3,4,6,7,8-Hexahydro-2H-pyrimido[1,2-A]pyrimidine) are described, but the results were poor. Crystallisation-induced asymmetric transformation using chiral cyclic amides, such as prolinamide, has not been reported.

### DESCRIPTION OF THE INVENTION

The present invention makes it possible for the resolution and racemisation of mandelic acids to progress effectively simultaneously in the same reactor vessel or reaction system as described below.

According to the invention there is provided a process for dynamically resolving an optionally substituted (R)- or (S)-mandelic acid from an enantiomeric mixture of said optionally substituted mandelic acid by salt formation with a chiral base (D)- or (L)-cyclic amide comprising the steps of:
(a) forming a resolving mixture in a solvent, or mixture of solvents, of
   (i) an enantiomeric mixture of an optionally substituted mandelic acid;
   (ii) a chiral base (D)- or (L)-cyclic amide, and optionally
   (iii) an additional racemising base;
      at an acid:total base (i.e. cyclic amide and optional additional racemising base) molar ratio of at least 1:1; provided that the cyclic amide base:acid molar ratio is at least 0.75:1; and wherein the resolving mixture may optionally contain water in the range of 2% to 15% (vol.) of solvent;
(b) heating the resolving mixture above ambient temperature and
(c) separating the respective optionally substituted (R)- or (S)-mandelic acid-cyclic amide salt.

The resolution may be started (according to the procedure disclosed in PCT application PCT/GB2004/004964) with, for example, 0.5 equivalents of D-prolinamide. Another 0.6 equivalents of D-prolinamide may then be added once the crystallisation has started, with the 0.1 equivalents excess of D-prolinamide acting as a base for racemisation. The ratio of the R- vs the S-enantiomer is typically about 85/15 after about 22 hours at 90° C., and the yield of (2R)-[3-chloro-5-(difluoromethoxy)phenyl](hydroxy)acetic acid after filtration and a slurry wash is about 73%.

In an alternative, the resolution may be started (according to the procedure disclosed in PCT application PCT/GB2004/004964) with, for example, 0.5 equivalents of D-prolinamide. Another 0.7 equivalents of D-prolinamide may then be added once the crystallisation has started, with the 0.2 equivalents excess of D-prolinamide acting as a base for racemisation. The ratio of the R- vs the S-enantiomer is typically about 85/15 after about 22 hours at 100° C., and the yield of the D-prolinamide salt of (2R)-[3-chloro-5-(difluoromethoxy)phenyl](hydroxy)acetic acid with 99% ee after filtration and a slurry wash is about 82%.

Alternatively, an excess equivalent of base (for example, 1.1 equivalents) may be added all at once at the start of the resolution.

Furthermore, a mixture of bases may be employed. For example, in this embodiment, a cyclic amide salt (as defined herein) is used to perform the resolution, whilst an alternative organic amine base (typically one with a pKa in the range 9-14, such as benzylamine, 1,8-diazabicyclo[5.4.0]undec-7-ene (DBU), 1,5-diazabicyclo[4.3.0]non-5-ene (DBN), 1,4-diazabicyclo[2.2.2]octane (Dabco), hexylamine, cyclohexylamine, dicyclohexylamine, piperidine, piperazine, ethylenediamine, phenethylamine, 2-aminoethanol, or 4-amino-1-butanol) is used to perform the racemisation. The ratio of the cyclic amide base:organic amine base may vary provided sufficient cyclic amide base is provided for resolution purposes (for example, 0.75-1.0 equivalents based on mandelic acid), and sufficient organic amine base is provided to effect racemisation (for example, 0.1-0.5 equivalents based on mandelic acid). The organic amine base may be added at the same time as the cyclic amide base, or after a suitable interval (to permit a degree of resolution to occur). Furthermore, the amount of cyclic amide base and organic amine base that is added may be added all at once, or in separate portions.

In a further alternative, the additional racemising base may be a carbonate or hydroxide of a Group I or Group II metal, such as sodium or potassium hydroxide; or potassium or magnesium carbonate.

In a further alternative, the mixture of mandelic acids to be resolved may be added in portions to the cyclic amide base (and optional alternative base). In this way, an excess equivalent of base is maintained during addition of the acid.

It is believed that the acid:total base (i.e. cyclic amide and optional additional racemising base) molar ratio should be at least 1:1 so that the acid is in the form of a salt/s during the process, and that the respective solubility of different salts permits separation of the respective (R)- or (S)-mandelic acid-cyclic amide salt.

In this specification, unless otherwise stated, the term “cyclic amide” includes optionally substituted forms thereof and includes, but is not limited to, proline amide, azetidine-2-carboxamide and piperidine-2-carboxamide as well as substituted forms thereof. Substitution may be on a ring nitrogen atom, by C₁₋₆ Alkyl, or on a suitable ring carbon atom by C₁₋₆ Alkyl or halo (for example, chloro, fluoro or bromo). Unsubstituted cyclic amides are preferred, but when substituted, substitution on a ring nitrogen atom or mono-substitution on a suitable ring carbon atom is preferred.

In this specification it is to be understood that, unless stated otherwise, when a (D) or (L) cyclic amide salt is drawn (as for example in formula II) then the cyclic amide may be optionally substituted on the nitrogen atom by C₁₋₆ Alkyl, or on a suitable ring carbon atom by C₁₋₆ Alkyl or halo (such as fluoro, chloro or bromo) as shown in formula I(x) below (wherein n is 0, 1 or 2; R₁ is H or C₁₋₆ Alkyl and X is H, halo or C₁₋₆ Alkyl) . . . .

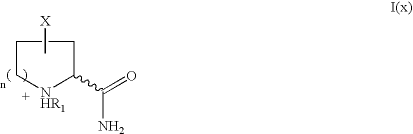

In this specification it is to be understood that an optionally substituted (D) cyclic amide as described herein has the (2R) stereochemistry shown in formula I(y) below (wherein n is 0, 1 or 2; R₁ is H or C₁₋₆ Alkyl and X is H, halo or C₁₋₆ Alkyl) . . . .

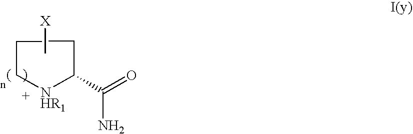

In this specification it is to be understood that an optionally substituted (L) cyclic amide as described herein has the (2S) stereochemistry shown in formula I(z) below, (wherein n is 0, 1 or 2; R₁ is H or C₁₋₆ Alkyl and X is H, halo or C₁₋₆ Alkyl) . . . .

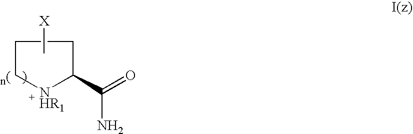

It is to be understood that all isomers within the definitions of chiral base cyclic amide disclosed herein are covered by the invention.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl.

Particularly, there is provided a process for resolving (R)- or (S)-optionally substituted mandelic acids from a (racemic) mixture of said optionally substituted mandelic acids by salt formation with a chiral base (D)- or (L)-cyclic amide, and racemisation of the unresolved enantiomer in the same process, comprising the steps:

(a) forming a mixture in a solvent, or mixture of solvents, of
(i) a (racemic) mixture of, optionally substituted, mandelic acid enantiomers;
(ii) a chiral base (D)- or (L)-cyclic amide, wherein the chiral base used is either (D) for separation of (R)-mandelic acids, or (L) for separation of (S)-mandelic acids, and optionally
(iii) an additional racemising organic amine base;
   at an acid:total base (i.e. cyclic amide and optional organic amine) molar ratio of at least 1:1; provided that the cyclic amide base is present in a molar ratio of at least 0.75; and wherein the mixture may optionally contain water in the range of 2% to 15% (vol.) of solvent;
   (b) heating the mixture above ambient temperature and
   (c) separating the respective (R)/(D) or (S)/(L) mandelic acid-cyclic amide salt.

Particularly, there is provided a process for resolving (R)- or (S)-substituted mandelic acids from a (racemic) mixture of said substituted mandelic acids by salt formation with a chiral base (D)- or (L)-cyclic amide, and racemisation of the unresolved enantiomer in the same process, comprising the steps:

(a) forming a mixture in a solvent, or mixture of solvents, of
(i) a (racemic) mixture of mandelic acid derivative enantiomers of formula I;

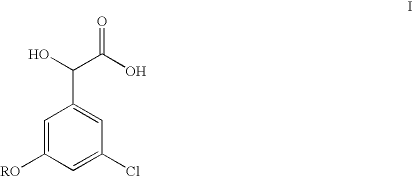

wherein R is selected from CHF₂, H, C₁₋₆ Alkyl, CH₂F, CHCl₂ and CClF₂;
(ii) either a chiral base (D)-cyclic amide or (L)-cyclic amide of formula I(x) wherein n is 0, 1 or 2; R₁ is H or C₁₋₆ Alkyl and X is H, halo or C₁₋₆ Alkyl,

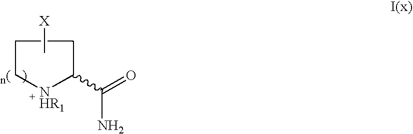

wherein the chiral base used is either (D) for separation of (R)-mandelic acids, or (L) for separation of (S)-mandelic acids; and optionally
(iii) an additional racemising organic amine base;
   at an acid:total base (i.e. cyclic amide and optional organic amine) molar ratio of at least 1:1; provided that the cyclic amide base is present in a molar ratio of at least 0.75; and wherein the mixture may optionally contain water in the range of 2% to 15% (vol.) of solvent;
   (b) heating the mixture above ambient temperature and
   (c) separating the respective (R)/(D) or (S)/(L) mandelic acid-cyclic amide salt of formula Ia;

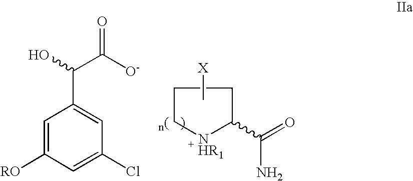

Particular mandelic acid-cyclic amide salts of formula IIa are of formula II;

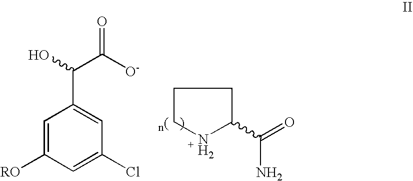

wherein R is selected from CHF₂, H, C₁₋₆ Alkyl, CH₂F, CHCl₂ and CClF₂; and n is 0, 1 or 2.

In one aspect of the process of the invention, the acid:total base molar ratio is 1:1.025 to 2.500, for example 1:1.10 to 1.50 (for example 1:1.10).

Statements that the cyclic amide base is present in a molar ratio of at least 0.75 mean that the cyclic amide base:acid molar ratio is at least 0.75:1.

It is to be understood that the molar ratios in this specification also cover experimental variation around these limits, e.g. ±0.005.

Suitable solvents for the process of the invention include, but are not limited by, the following ethyl acetate, iso-propyl acetate, n-butyl acetate (in general, (1-4C) acetates may be used), MIBK, DMF, DMSO, DMA, dioxane, N-methylpyrrolidinone, acetonitrile, acetone, 2-butanone, 4-methyl-2-pentanone, tert-butyl methyl ether, ethanol, 2-propanol (in general, any higher alcohol may be used), heptane, iso-octane or a mixture of any of these solvents.

Solvents other than ethyl acetate or 4-methyl-2-pentanone (MIBK, methyl isobutyl ketone) may be used, and are suitable for the formation of (S)-3-chloro,5-difluoro-methoxy mandelic acid.L-prolinamide salt. These solvents include acetonitrile, acetone, 2-butanone (MEK, methyl ethyl ketone), tert-butyl methyl ether (TBME), 2-propanol and ethanol. It is expected that these solvents can also be applied in formation of the (R)-3-chloro,5-difluoro-methoxy mandelic acid.D-prolinamide salt.

The above-mentioned solvents may be used as pure solvents, or as mixtures with other solvents from those mentioned above. Furthermore, the solvent or solvent mixture may optionally contain water (suitably in an amount from 2% to 15% v/v). A preferred solvent is one with a boiling point above 70° C. to 80° C. Acetate solvents (especially iso-propyl acetate or n-butyl acetate) or MIBK are specifically preferred.

The process of the invention is performed at a temperature above ambient temperature (typically 20° C.) to ensure that racemisation proceeds at an appropriate rate. A suitable temperature depends on the solvent system selected and is, for example, above 50° C. to 70° C., preferably above 70° C., and up to the reflux temperature of the mixture.

In another aspect, there is provided a process of the invention which forms an isolated mandelic acid cyclic amide salt of formula III;

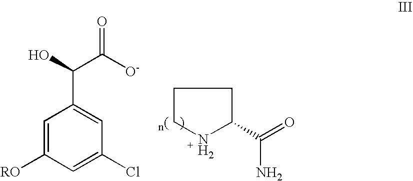

wherein R is selected from CHF₂, H, C₁₋₆ Alkyl, CH₂F, CHCl₂ and CClF₂; and n is 0, 1 or 2.

In one embodiment of this aspect there is provided a process wherein R of Formula III is CHF₂, and n of Formula III is 1, represented by Formula VI;

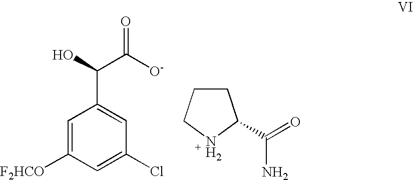

In another aspect, there is provided a process of the invention which forms an isolated mandelic acid cyclic amide salt of formula IV;

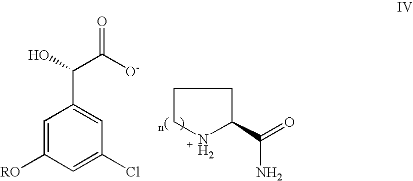

wherein R is selected from CHF₂, H, C₁₋₆ Alkyl, CH₂F, CHCl₂ and CClF₂; and n is 0, 1 or 2.

In one embodiment of this aspect, there is provided a process, wherein R of Formula IV is CHF₂, and n of Formula IV is 1, represented by Formula VII;

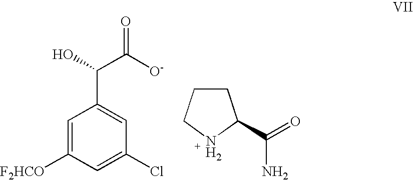

In another aspect, there is provided a process of the invention, wherein R of Formula I is CHF₂, represented by Formula V;

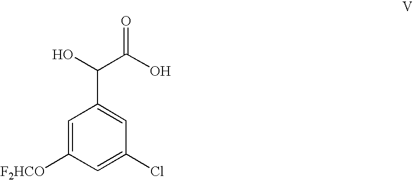

In the above aspects and embodiments, the cyclic amide used may be optionally substituted on the nitrogen atom by C₁₋₆ Alkyl, or on a suitable ring carbon atom by C₁₋₆ Alkyl or halo (such as fluoro, chloro or bromo) as shown for formula I(x) above.

The (racemic) mandelic acid derivative/cyclic amide/optional additional organic amine base and solvent (for example, ethyl acetate) mixture in step (a) of the processes may be optionally heated to reflux. The presence of water (in the range of 2% to 15% (vol.) of solvent) is preferred, and the heating of the mixture may be followed by addition of the water to obtain a suspension. This suspension is normally stirred at reflux for 10 minutes before cooling and separating the desired mandelic acid-cyclic amide salt.

The concentration of (racemic) mandelic acid derivative in the solvent mixture is usually in the range of 0.25-2.5 mmol per ml of solvent. Preferably, the (racemic) mandelic acid derivative is added at a concentration range of 0.25-2.0 mmol per ml of solvent. Particularly preferred is when the (racemic) mandelic acid derivative is added at a concentration range of 0.25-1.25 mmol per ml of solvent.

The isolated salt may be dissolved in a mixture of HCl and solvent (such as ethyl acetate) followed by separation of the organic layer and concentrating said organic layer to dryness to obtain the resolved mandelic acid derivative. Preferably, the mixture of HCl and solvent is a 1:1 (vol.) mixture of 1M HCl and solvent. The resolved mandelic acid derivative may be analysed by conventional chiral HPLC techniques.

Alternatively, (S)-3-chloro,5-difluoro-methoxy mandelic acid.L-prolinamide salt may be isolated and then a different salt of (R)-3-chloro,5-difluoro-methoxy mandelic acid isolated from the mother liquors (such as the triethanolamine salt).

The said mandelic acid cyclic amide salts represented by the Formulas II, III, IV, VI and VII are obtainable by the processes of the present invention.

Also provided are the products obtainable by the processes described within this specification and within any of the Examples disclosed herein.

There is a need for a more convenient and more economically efficient process for the manufacturing of large scale quantities of high quality (pure) resolved mandelic acid derivatives, where factors like costs, manufacturing time, use of more environmentally friendly solvents etc. are vital for commercial application. The present invention provides for such a process. The processes of the invention use an improved process for the manufacture of resolved mandelic acid derivatives in which non-expensive raw materials and thermally safe work up conditions are used to achieve these quality resolved mandelic acid derivatives ready to use in further chemical processing.

The invention further provides the use of a mandelic acid-cyclic amide salt according to the invention in the manufacture of pharmaceutical products; the use of a mandelic acid-cyclic amide salt according to the invention as chemical intermediates and the use of a mandelic acid cyclic amide salt according to the invention as chemical intermediates in manufacture of pharmaceutical products (for example for use in treating cardiovascular diseases).

In this specification the term “racemic mixture” may include mixtures of enantiomers in ratios other than, as well as, a 50:50 mixture of R:S enantiomers (for example from 99:1 to 1:99). A particular process of the invention begins with a 50:50 mixture of enantiomers. The process may involve differing mixtures of enantiomers at various stages (including, but not limited to 50:50 mixtures). The term “racemisation” covers the conversion of an unresolved enantiomer into a mixture containing the enantiomer to be resolved.

The phrase “e.e.” denotes an abbreviation for enantiomeric excess and is defined as the mole fraction denoting the enantiomers in a mixture:

% e.e.=([R]−[S])/([R]+[S])

where [R] and [S] are the concentrations of the (R)- and (S)-enantiomers. In a reaction a chiral compound is often obtained as a mixture of enantiomers. If, for example, 80% of the (R)-enantiomer is formed and 20% of the (S)-enantiomer then the e.e. is: (80-20)/(80+20)=60%.

### EXAMPLES

The present invention is described in more detail in the following non-limiting Examples.

### Example 1

### Dynamic Resolution Using L-Prolinamide

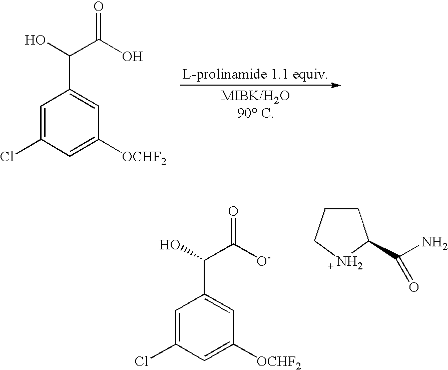

Methyl iso-butyl ketone (MIBK; 4.3 ml/g of mandelic acid) was added to the mandelic acid (1 eq.) at ambient temperature. Stirring was started and the solution was heated to 80° C. A solution of L-prolinamide (0.5 eq.) in water (3 molar equivalent/mandelic acid) was added and crystallisation started soon after. After half an hour additional MIBK (same as before) was added and then a solution of L-prolinamide (0.6 eq.) in water (3 molar equivalent/mandelic acid). The suspension was stirred at 80° C. for 4 hours then at 90° C. for 21 hours. The suspension was cooled to 0° C. over 1¾ hours. The substance was isolated by filtration, washed with MIBK and then dried (crude yield 75%). If the optical purity and the assay are not satisfactory, a series of slurry wash experiments in MIBK with varying water content (0-15% w/w) has shown that both the optical purity and the assay (physical content purity) can be improved. Extrapolation of the results indicated that a slurry wash in MIBK with 20% w/w of H₂O should give a substance with 99% ee and with an assay of 100% (the yield of the (S)-mandelic acid.L-prolinamide salt would then be about 73%).

The above experiment using L-prolinamide may be repeated using D-prolinamide to obtain the (R)-enantiomer of the mandelic acid.

The yield from one batch of this dynamic resolution process is comparable with the yield from three cycles of the resolution/racemisation process disclosed in PCT application PCT/GB2004/004964 (and the quality/purity of the material is comparable).

### Example 2

### Dynamic Resolution Using D-Prolinamide

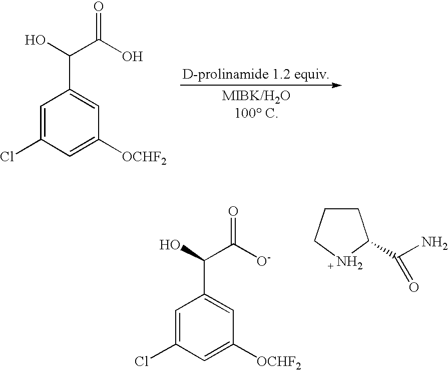

Methyl iso-butyl ketone (MIBK; 3.87 ml/g of mandelic acid) was added to the mandelic acid (1 eq.) at ambient temperature. Stirring was started and the solution was heated to 80° C. A solution of D-prolinamide (0.5 eq.) in MIBK (0.43 ml/g of mandelic acid) and water (3 molar equivalent/mandelic acid) was added and crystallisation started soon after. After half an hour additional MIBK (3.87 ml/g of mandelic acid) was added and then a solution of D-prolinamide (0.7 eq.) in MIBK (0.43 ml/g of mandelic acid) and water (3 molar equivalent/mandelic acid). The suspension was stirred at 100° C. for 22 hours. The suspension was cooled to 0° C. over 2.25 hours. The substance was isolated by filtration, washed with MIBK and then dried (crude yield 84.9%). The ee of the crude D-prolinamide salt of the (R)-enantiomer of the mandelic acid was 94.32%.

If the optical purity and the assay of the salt are not satisfactory, a series of slurry wash experiments in a number of solvents has shown that both the optical purity and the assay (physical content purity) can be improved. A slurry wash in acetone, for example, gave the (R)-mandelic acid.D-prolinamide salt with 99.1% ee. The yield including the slurry wash was 81.8%. With 2-butanone (MEK) as solvent for the slurry wash, the (R)-mandelic acid.D-prolinamide salt was obtained with 96.0% ee in 83.9% yield. Other solvents or solvent mixtures which can be used for the slurry wash are MIBK with 20% w/w H₂O, acetonitrile, and 2-propanol.

The above experiment using D-prolinamide may be repeated using L-prolinamide to obtain the (S)-enantiomer of the mandelic acid.

The yield from one batch of this dynamic resolution process is higher than the yield from three typical cycles of the resolution/racemisation process disclosed in PCT application PCT/GB2004/004964 (and the quality/purity of the material is comparable).

### REFERENCE EXAMPLES

### Racemisation of Mother Liquor

### Reference Examples 1-3

In these Reference Examples the following method was used, with volumes and amounts as outlined in Table 1.

The racemic mandelic acid derivative 3-chloro,5-difluoro-methoxy mandelic acid and (D)-proline amide were added to ethyl acetate saturated in water (8.1% water in ethyl acetate). The mixture was heated to reflux and stirred for 10 minutes at reflux. The thin suspension was cooled to 23° C. over 13 hours followed by further cooling to 18° C. over 40 minutes. The suspension was filtered and washed with ethyl acetate (3×30 ml) to give the salt. A is sample was dissolved in a 1:1 mixture of 1 M HCl and ethyl acetate. The organic layer was separated, concentrated to dryness and analysed by chiral HPLC (for suitable methodology, see Reference Example 11A). This showed a high degree of purity of the “correct” enantiomer (see Table 1), (R)-3-chloro,5-difluoro-methoxy mandelic acid.

TABLE 1

| Reference | | | | | Water/ | mmol MA/ | |
|---|---|---|---|---|---|---|---|
| Example | mmol | mmol | Eq. | EtOAc | EtOAc | ml water- | e.e. |
| No. | MA¹ | PA | PA | (ml) | (%) | EtOAc | (%) |
| 1 | 1.16 | 0.57 | 0.49 | 0.97 | 8.1 | 1.20 | 84.2 |
| 2 | 1.16 | 0.57 | 0.49 | 0.51 | 8.1 | 2.27 | 95.3 |
| 3 | 1.09 | 0.53 | 0.49 | 0.67 | 8.1 | 1.63 | 90.6 |

MA = racemic mandelic acid derivative, 3-chloro,5-difluoro-methoxy mandelic acid.
PA = (D)-proline amide.
Eq. PA = Amount of equivalents of (D)-proline amide compared to racemic mandelic acid derivative.
EtOAc = ethyl acetate, as solution saturated in water.
Water/EtOAc (%) = concentration of water in ethyl acetate.
mmol MA/ml water-EtOAc = concentration range of racemic mandelic acid derivative per ml of ethyl acetate and water.
e.e. (%) = enantiomeric excess defined as the % mole fraction denoting the enantiomers in a mixture.
¹Corrected for purity, i.e. initially 86% pure racemic mandelic acid derivative.

### Reference Examples 4-9

In these Reference Examples the following method was used, with volumes and amounts as outlined in Table 2.

The racemic mandelic acid derivative 3-chloro,5-difluoro-methoxy mandelic acid and (D)-proline amide were added to ethyl acetate and the mixture heated to reflux. At reflux, water was added and the mixture was stirred for another 10 minutes at reflux. The thin suspension was allowed to cool to 18° C. over 3 hours (in Reference Examples 4-8; 4 hours in Reference Example 9). The suspension was filtered and washed with ethyl acetate (3×30 ml) to give the salt. The salt was dissolved in a 1:1 mixture of 1 M HCl and ethyl acetate. The organic layer was separated, concentrated to dryness and analysed by chiral HPLC (for suitable methodology, see Reference Example 11A). This showed a high degree of purity of the “correct” enantiomer (see Table 2), (R)-3-chloro,5-difluoro-methoxy mandelic acid.

To exemplify in more detail, the following scheme was used in Reference Example 6:

The racemic mandelic acid derivative 3-chloro,5-difluoro-methoxy mandelic acid (26.18 g, 93.3 mmol, 1 eq, 90% pure according to HPLC) and (D)-proline amide (4.80 g, 42 mmol, 0.45 eq) were added to ethyl acetate (54.5 ml) and the mixture heated to reflux. At reflux, 5.5 ml of water was added and the mixture stirred for another 10 minutes at reflux. The thin suspension was allowed to cool to 18° C. over 3 hours. The suspension was filtered and washed with ethyl acetate (3×30 ml) to give 8.6 g of the salt. A sample was dissolved in a 1:1 mixture of 1 M HCl and ethyl acetate. The organic layer was separated, concentrated to dryness and analysed by chiral HPLC. This showed 98.2% of the “correct” (R)-enantiomer. From the mother liquor more material crystallised, which was filtered, washed and dried. This gave another 1.6 g of the salt. The free (R)-mandelic acid was analysed by HPLC (for suitable methodology, see Reference Example 11A) and contained 99.0% of the “correct” enantiomer.

TABLE 2

| | | | | | | | mmol | |
|---|---|---|---|---|---|---|---|---|
| Reference | | | | | | Water/ | MA/ml | |
| Example | mmol | mmol | | EtOAc | Water | EtOAc | water- | e.e. |
| No. | MA¹ | PA | Eq. PA | (ml) | (ml) | (%) | EtOAc | (%) |
| 4 | 5.96 | 2.9 | 0.49 | 3.7 | 0.30 | 7.5 | 1.61 | 99.2 |
| 5 | 10.45 | 5.1 | 0.49 | 6.4 | 0.52 | 7.5 | 1.51 | 98.9 |
| 6 | 93.30 | 42.0 | 0.45 | 54.5 | 5.50 | 9.2 | 1.40 | 98.7 |
| 7 | 155.31 | 77.7 | 0.50 | 91.5 | 10.20 | 10.0 | 1.53 | 99.0 |
| 8 | 76800 | 38400 | 0.50 | 66800 | 4600 | 6.4 | 1.08 | 98.2 |
| 9² | 42240 | 21120 | 0.50 | 33000 | 2500 | 7.0 | 1.19 | 99.6 |

MA = racemic mandelic acid derivative 3-chloro,5-difluoro-methoxy mandelic acid.
PA = (D)-proline amide.
Eq. PA = Amount of equivalents of proline amide compared to racemic mandelic acid derivative
EtOAc = ethyl acetate in ml.
Water/EtOAc (%) = concentration of water in ethyl acetate.
mmol MA/ml water-EtOAc = concentration range of racemic mandelic acid derivative per ml of ethyl acetate and water.
e.e. (%) = enantiomeric excess defined as the % mole fraction denoting the enantiomers in a mixture.
¹Corrected for purity, i.e. initially 85-90% pure racemic mandelic acid derivative.
²The suspension was allowed to cool to 18° C. over 4 hours.

### Reference Example 10

The racemic mandelic acid derivative 3-chloro,5-difluoro-methoxy mandelic acid (0.2 g, 0.79 mmol) and (L)-proline amide (0.05 g, 0.48 mmol, 0.6 eq,) were added to 1 ml dioxane and the mixture heated to 90° C. During heat-up a thick suspension was formed. The suspension was filtered and (S)-mandelic acid liberated by extractive work up using 1 M HCl and ethyl acetate. 0.05 g enantiomer of ee: 92% was obtained.

### Reference Example 11A

### Racemisation of Mother Liquor

The mother liquor, in ethyl acetate, from the resolution process (for example, from any of Reference Examples 1-9 above), containing the “wrong” mandelic acid enantiomer in excess (3.35 kg, 3.53 L, corresponds to 0.462 kg mandelic acid, 1.83 mol) was concentrated under reduced pressure at 50-55° C. to a volume of 2.78 L. The solution was extracted at 15-25° C. with 10% aqueous hydrochloric acid (0.62 kg, 1.69 mol, 0.92 eq) to remove D-prolinamide. The organic solution was washed with deionised water (0.58 kg) after which phase inversion occurred with the organic phase below the aqueous phase. Sodium chloride (0.030 kg) was added to invert the phases again and the phases were separated. The organic phase was washed with 8.7% aqueous NaHCO₃ (0.71 kg, 0.74 mol, 0.40 eq). The organic phase was concentrated as much as possible under reduced pressure at 50-60° C. The remaining residue (0.483 kg) had a chemical purity of 76.5% as determined by HPLC and an optical purity for the S-enantiomer of 81% as determined by chiral HPLC. The residue was dissolved in methanol (1.33 kg, 1.67 L) and 30% aqueous potassium hydroxide (0.84 kg, 4.46 mol, 2.43 eq) was added at 25-40° C. The mixture was heated to 68-75° C. and stirred for approximately 3.5 hours until complete racemisation had occurred according to chiral HPLC. Methanol was distilled off under reduced pressure at 40-50° C. Dichloromethane (1.35 kg, 1.02 L) and water (0.20 kg) were added to the aqueous solution and the mixture was cooled to 0-5° C. 20% Aqueous hydrochloric acid (1.17 kg, 1.10 L, 6.41 mol, 3.50 eq) was added within 20 minutes to the stirred two-phase mixture at T=0-20° C. (exothermic reaction, pH=1). The mixture was stirred over a period of about 10 minutes at 20-25° C. until the precipitated oily product was dissolved completely in dichloromethane. The phases were separated and the aqueous solution was extracted with dichloromethane (0.53 kg, 0.40 L). The combined organic phases were washed with water (0.48 kg) and concentrated under reduced pressure at 40-50° C. This gave 0.443 kg of an oily product with a HPLC purity of 97.1 area %.

The HPLC conditions used for determination of the purity of the MAPA salt by HPLC were:

| Column: | Symmetry Shield RP8, 2.1 × 50 mm, 3.5 μm, Waters | | |
|---|---|---|---|
| Flow rate: | 0.5 mL/min. | | |
| Detection: | UV, 220 nm | | |
| Volume injection: | 15 μL | | |
| Temperature | 20° C. | | |
| column: | | | |
| “Running time”: | 35 min.; Post time: 5 min. | | |
| Mobile phase: | A: 50 mL acetonitrile + 200 mL ammonium | | |
| | dihydrogenphosphate buffer + 750 mL pure water | | |
| | B: 800 mL acetonitrile (HPLC-grade) + 200 mL | | |
| | ammonium dihydrogenphosphate buffer | | |
| | Time (min) | % Phase A | % Phase B |
| Gradient: | 0 | 90 | 10 |
| | 5 | 90 | 10 |
| | 30 | 10 | 90 |
| | 35 | 2 | 98 |

The HPLC conditions used for determination of the optical purity of the MAPA salt by HPLC were:

| | |
|---|---|
| Column: | Chiralpak AD, 250 × 4.6 mm, DAICEL |
| Flow rate: | 1.0 mL/min |
| Detection: | UV, 215 nm |
| Volume injection: | 10 μL |
| Temperature column: | 20° C. |
| “Running time”: | 30 min |
| Mobile phase: | n-Hexane/2-propanol/trifluoroacetic |
| | acid = 900 mL/100 mL/1 mL |

The resulting racemate may again be used in the process of the invention to isolate more of the desired enantiomer, for example according to the following Reference Example.

### Reference Example 11B

### Resolution of the Mandelic Acid Obtained after Racemisation

A solution of the racemic mandelic acid (obtained after the first racemisation) in ethyl acetate (1.433 kg of a 29.9% (w/w) solution, 0.429 kg racemic mandelic acid, 1.698 mol, 1.00 eq) was filtered and added within 30 minutes to a stirred solution of D-prolinamide (0.095 kg, 0.853 mol, 0.49 eq) in ethyl acetate (0.407 kg, 0.452 L) as well as water (0.153 kg) at 72-75° C. After the addition was completed a clear solution was obtained. The mixture was cooled to 58° C. within 45 min. No crystallisation was observed. The mixture was cooled further to 0-2° C. within 2.5 hours. The salt started to precipitate at approximately 55° C. After stirring for a further hour at 0-2° C., the solid was filtered off and washed twice with a pre-cooled (0-5° C.) mixture of ethyl acetate/water=9:1 (w/w, 2×0.20 kg). A wet, off-white powder (0.264 kg) was obtained in 99.3% purity and 97.6% optical purity.

If necessary, the optical purity can be further improved by slurrying the product with ethyl acetate/water and filtering. For example, the optical purity can be improved further by the following re-work procedure.

### Reference Example 11C

### Re-Work Procedure

The wet mandelic acid D-prolinamide salt (0.264 kg) was suspended in a mixture of ethyl acetate (1.00 kg, 1.11 L) and water (0.10 kg). The suspension was heated to 73-75° C. and stirred for 30 minutes at this temperature. The suspension was cooled to 3-5° C. within 2 hours and then stirred for another hour at this temperature. The solid was filtered off and washed twice with a pre-cooled (0-5° C.) mixture of ethyl acetate/water=9:1 (w/w, 2×0.38 kg). The white solid was dried under reduced pressure (10 mbar) at 35-40° C. until the mandelic acid.D-prolinamide salt had constant weight. This gave 0.225 kg of product (73.9%, based on D-prolinamide) with a chemical purity of >99% and optical purity of >99%.

This racemisation-resolution procedure can be repeated, for example twice. Furthermore, the D- or L-prolinamide may be recycled using conventional extraction techniques.

### Reference Example 12

### Different Salts

Once the mandelic acid enantiomers are separated then the desired enantiomer can be isolated as a different salt suitable for further processing. Depending upon which mandelic acid enantiomer is required, such a different salt may be isolated either from the prolinamide salt, or from the mother liquors remaining after the prolinamide salt has been filtered off.

Thus, for example, (R)-3-chloro,5-difluoro-methoxy mandelic acid.D-prolinamide salt may be isolated and then converted into a different salt for further processing. The mother liquors can then be racemised for recycling, for example as described before.

Alternatively, (S)-3-chloro,5-difluoro-methoxy mandelic acid.L-prolinamide salt may be isolated and then a different salt of (R)-3-chloro,5-difluoro-methoxy mandelic acid isolated from the mother liquors (such as the triethanolamine salt). The (S)-3-chloro,5-difluoro-methoxy mandelic acid.L-prolinamide salt may then be used for racemisation and recycling.

(R)-3-chloro,5-difluoro-methoxy mandelic acid ((2R)-[3-chloro-5-(difluoromethoxy)-phenyl](hydroxy)acetic acid) is a useful intermediate, but the free acid compound has a low melting point (52° C.) and is hard to crystallise. Furthermore, (R)-3-chloro,5-difluoro-methoxy mandelic acid is very soluble compared to the unsubstituted mandelic acid. Although 3-chloro,5-difluoro-methoxy mandelic acid is capable of forming salts with, for example, α,α-diphenyl-D-prolinole, such salts are not satisfactory for large-scale manufacturing purposes (having low yield and low enantiomeric excess).

In PCT application PCT/GB2004/00496 the Examples describe the isolation of, for example, (R)-3-chloro,5-difluoro-methoxy mandelic acid from a racemic mixture by resolution with D-prolinamide. These cyclic amide resolving salts are expensive, and thus cheaper salts are of further interest to permit even more efficient large-scale manufacturing.

In PCT application PCT/GB2004/004964 further new salts are provided of substituted mandelic acids. The discovery of such salts provides an efficient, inexpensive isolation of mandelic acids as a solid, thereby creating opportunities for economic enantioselective processes and for improvements of the process using resolution with, for example, D-prolinamide.

Enantioselective routes to (R)-3-chloro,5-difluoro-methoxy mandelic acid are also of interest, and in such cases an efficient, inexpensive salt of the mandelic acid is attractive. Preferably the salt should be crystalline, enhance the enantiomeric purity upon formation and be directly useable in a subsequent (coupling) reaction.

The Reference Examples below from PCT application PCT/GB2004/004964 describe the preparation of the triethanolamine salt of (R)-3-chloro,5-difluoro-methoxy mandelic acid.

### Reference Example 12

### Triethanolamine Salt

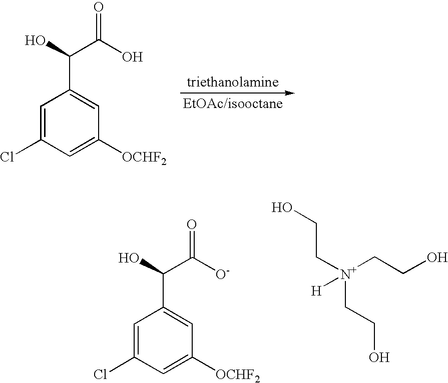

Triethanolamine (211.8 μl, 1.564 mmol) was added to a 0.356 M solution of the (R)-mandelic acid (0.359 g, 1.422 mmol; prepared from the (R)-MA-(D)-PA salt using HCl(aq), and water washing) in ethyl acetate at ambient temperature. The addition was accompanied by a weak exotherm. The solution was heated to 66° C. and isooctane added until the solution started to turn cloudy. The solution was cooled slowly to ambient temperature overnight. The solution was then cooled to 0° C. and the salt precipitated after 1½ hours stirring at 0° C. The suspension was stored in the refrigerator overnight, filtered, washed with EtOAc/isooctane 1.46:1 (2×1.23 ml), then vacuum dried at 40° C. to give 0.500 g (1.244 mmol, 88%) of the crystalline (R)-3-chloro,5-difluoro-methoxy mandelic acid.triethanolamine salt (melting-point (MP)=68° C.). The crystallinity of the triethanolamine salt of the (R)-mandelic acid was confirmed by DSC (endotherm onset=68° C.) and XRPD. The following XRPD d-values and intensities were obtained:

| d-value | Relative |
|---|---|
| (Å) | intensity |
| 7.3 | m |
| 6.9 | m |
| 6.1 | s |
| 5.6 | vs |
| 5.4 | m |
| 5.2 | m |
| 4.60 | m |
| 4.45 | m |
| 4.33 | m |
| 4.11 | m |
| 3.80 | s |
| 3.72 | vs |
| 3.64 | s |
| 3.59 | m |
| 3.48 | m |
| 3.46 | m |
| 3.35 | m |
| 3.31 | m |
| 3.24 | m |
| 3.09 | m |
| 3.05 | m |
| 2.92 | m |
| 2.79 | m |
| 2.60 | m |

The main, reproducible peaks have been tabulated using the following definitions . . .

| | |
|---|---|
| vs (very strong): | >50% rel. int. |
| s (strong: | 28-50% rel. int. |
| m (medium): | 9-28% rel. int. |
| w (weak): | 4-9% rel. int. |
| vw (very weak): | <4% rel. int. |

The relative intensities are derived from diffractograms measured with variable slits.

X-ray powder diffraction analysis (XRPD) was performed on samples prepared according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London; or Klug, H. P. & Alexander, L. E. (1974), X-ray Diffraction Procedures, John Wiley and Sons, New York. X-ray analyses were performed using a PANalytical X'Pert PRO MPD diffractometer. The sample was analysed with, and without, internal reference. The measured peak values were adjusted and thereafter calculated into d-values.

Differential scanning calorimetry (DSC) was performed using a PerkinElmer DSC7 instrument, according to standard methods, for example those described in Höhne, G. W. H. et al (1996), Differential Scanning Calorimetry, Springer, Berlin. DSC onset temperatures may vary in the range ±5° C. (e.g. ±2° C.), and XRPD distance values may vary in the range ±2 on the last given decimal place.

### Reference Example 12

### Enantiomeric Selectivity of the Conglomerate Triethanolamine Salt

The triethanolamine salt of 3-chloro-5-difluoromethoxy mandelic acid is particularly interesting as it occurs as a crystalline conglomerate. This makes it possible to improve the enantiomeric excess of (R)-3-chloro,5-difluoro-methoxy mandelic acid as product from an enantioselective process.

There is a distinct difference between a conglomerate and a racemic compound. Looking at a 50:50 mixture of both enantiomers, a conglomerate consists of a mixture of crystals of the two enantiomers in equal amounts. Although in bulk the conglomerate is optically neutral, the individual crystals contain only the R or S-enantiomer. This is in contrast to a racemic compound where the individual crystals contain equal amounts of both enantiomers and the racemic crystals form a perfectly ordered array of R and S molecules. Racemic compounds and conglomerates can be distinguished by determination of their melting point diagrams (phase diagrams) or by using powder X-ray diffraction or solid state IR spectroscopy; the data of pure enantiomers are identical with the data of the conglomerate, but different from that of a racemic compound.

For the triethanolamine salt of 3-chloro-5-difluoromethoxy mandelic acid, being a conglomerate makes it possible to isolate the triethanolamine salt of the (R)-mandelic acid from an enantiomerically enriched mixture of the mandelic acid by direct crystallisation. The maximum theoretical yield can be calculated by: 100-100×(amount of the wrong enantiomer present in the sample+same amount of the desired enantiomer)/total amount of solid. For example, starting with 95% w/w of the desired enantiomer, the maximum yield is 90%. Starting with 90% w/w of the desired enantiomer, the maximum yield is 80%, etc. (R)-3-chloro-5-difluoromethoxymandelic acid with an e.e. of 90% can, for example, be the product of an enantioselective process.

### Reference Example 12-1

Racemic 3-chloro-5-difluoromethoxy mandelic acid (51.25 mg, 0.203 mmol) was added to a 0.351 M solution of the (R)-mandelic acid (0.607 g, 2.405 mmol; prepared from the (R)-MA-(D)-PA salt using HCl(aq), and water washing) in ethyl acetate at ambient temperature. The enantiomeric excess of the (R)-mandelic acid in the solution was determined to be 92.4% by chiral HPLC analysis (performed as in Reference Example 11 above). Triethanolamine (0.417 g, 2.739 mmol) was added to the solution at 23° C. The temperature rose to 25° C. upon the addition. The solution was heated to 70° C. At 70° C., isooctane (1.5 ml) was added and the solution was seeded with a few granules of the triethanolamine salt of (R)-3-chloro-5-difluoromethoxy mandelic acid (99.8% ee; see Reference Example 12). The solution was cooled to 65° C. and since crystallization had not started the seeding was repeated. The solution was cooled to 26° C. over 3 hours, but as there was still no precipitation of the salt, the solution was heated again to 70° C., seeded and then allowed to cool. Finally, the crystallization started at 58° C. after another seeding. The suspension was cooled to ambient temperature and left to stir overnight. A sample was filtered off the next morning, the optical purity of which was determined to be 98.1% ee by chiral HPLC analysis (see Reference Example 11). The bulk of the suspension was cooled to, and stirred at 1° C. for 2¼ hours. The salt was isolated by filtration, washed with EtOAc/isooctane 2.5:1 (2×2.07 ml) and vacuum dried at 40° C. overnight to give the triethanolamine salt of (R)-3-chloro-5-difluoromethoxy mandelic acid as a white powder (0.897 g, 88.8%). The optical purity of the salt was determined to be 99.5% ee by chiral HPLC analysis (see Reference Example 11).

### Reference Example 12-2

Racemic 3-chloro-5-difluoromethoxy mandelic acid (371.29 mg, 1.470 mmol) was added to a 0.351 M solution of (R)-mandelic acid (3.500 g, 13.856 mmol; prepared from the (R)-MA-(D)-PA salt using HCl(aq), and water washing) in ethyl acetate at ambient temperature. The enantiomeric excess of (R)-mandelic acid in the solution was determined to be 91.1% by chiral HPLC analysis (see Reference Example 11). Triethanolamine (2.566 g, 16.856 mmol) was added to the solution at 23° C. The temperature rose to 29° C. upon the addition. The solution was heated to 70° C. At 70° C. isooctane (8.6 ml) was added and the solution was seeded with a few granules of the triethanolamine salt of (R)-3-chloro-5-difluoromethoxy mandelic acid (99.8% ee; see Reference Example 12). The solution was cooled to 65° C. and since crystallization had not started the seeding was repeated. The solution was cooled by stages and seeded four more times. Finally at about 40° C. the salt crystallized. The suspension was cooled to room temperature and left to stir overnight. A sample was filtered off the next morning, the optical purity of which was determined to be 97.0% ee by chiral HPLC analysis (see Reference Example 11). The bulk of the suspension was cooled to and stirred at 1° C. for 2¾ hours. The salt was isolated by filtration, washed with EtOAc/isooctane 4:1 (2×7.5 ml) and vacuum dried at 40° C. overnight to give the triethanolamine salt of (R)-3-chloro-5-difluoromethoxy mandelic acid as a white powder (5.451 g, 92.0%). The optical purity of the salt was determined to be 98.7% ee by chiral HPLC analysis (see Reference Example 11).

It is to be noted that any of the salts described herein may be in the form of polymorphs, solvates or hydrates, and such forms are also covered by the invention. Also covered by the invention are any tautomers of the mandelic acid derivatives described herein.

1. A process for dynamically resolving an optionally substituted (R)- or (S)-mandelic acid from an enantiomeric mixture of said optionally substituted mandelic acid by salt formation with a chiral (D)- or (L)-cyclic amide base comprising the steps of:
(a) forming a resolving mixture in a solvent, or mixture of solvents, of
(i) an enantiomeric mixture of an optionally substituted mandelic acid;
(ii) a chiral b (D)- or (L)-cyclic amide baser and optionally
(iii) an additional racemising base;
wherein the acid:total of cyclic amide base and optional additional racemising base molar ratio is at least 1:1; provided that the cyclic amide base:acid molar ratio is at least 0.75:1; and wherein the resolving mixture may optionally contain water in the range of 2% to 15% by volume of solvent;
(b) heating the resolving mixture above ambient temperature; and
(c) separating the respective optionally substituted (R)- or (S)-mandelic acid-cyclic amide salt.

2. The process according to claim 1, wherein the chiral (D)- or (L)-cyclic amide base is of formula I(x);
wherein
n is 0, 1 or 2;
R₁ is H or C₁₋₆ alkyl; and
X is H, halo or C₁₋₆ alkyl,

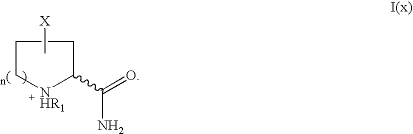

3. The process according to claim 1, for resolving (R)- or (S)-optionally substituted mandelic acids from a mixture of said optionally substituted mandelic acid enantiomers by salt formation with a chiral (D)- or (L)-cyclic amide base, and racemisation of the unresolved enantiomer in the same process, comprising the steps:
(a) forming a mixture in a solvent, or mixture of solvents, of
(i) a mixture of optionally substituted mandelic acid enantiomers;
(ii) a chiral (D)- or (L)-cyclic amide base, wherein the chiral base used is either (D) for separation of (R)-mandelic acids, or (L) for separation of (S)-mandelic acids; and optionally
(iii) an additional racemising organic amine base;
wherein the acid:total of cyclic amide base and optional organic amine base molar ratio is at least 1:1; provided that the cyclic amide base is present in a molar ratio of at least 0.75; and
wherein the mixture may optionally contain water in the range of 2% to 15% by volume of solvent;
(b) heating the mixture above ambient temperature; and
(c) separating the respective (R)/(D) or (S)/(L) mandelic acid-cyclic amide salt.

4. The process according to claim 1, wherein the base or bases are added in more than one portion and after the first portion, any additional portion may optionally be added after the heating step (b).

5. The process according to claim 1, wherein the additional racemising organic base is added before the chiral amide base.

6. The process according to claim 1, wherein the additional racemising organic base is added after the chiral amide base.

7. The process according to claim 1, wherein the acid:total base molar ratio is 1:1.025 to 1:2.500.

8. The process according to claim 1, wherein the cylic amide base is used alone.

9. The process according to claim 1, wherein the cylic amide base is used at an acid:base molar ratio of 1:0.75.

10. The process according to claim 1, wherein the temperature is above 50° C. to 70° C.

11. The process according to claim 1, for resolving (R)- or (S)-substituted mandelic acids from a mixture of the substituted mandelic acid enantiomers by salt formation with a chiral (D)- or (L)-cyclic amide base, and racemisation of the unresolved enantiomer in the same process, comprising the steps:
(a) forming a mixture in a solvent, or mixture of solvents, of
(i) a mixture of mandelic acid derivative enantiomers of formula I; 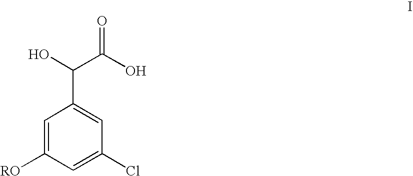
wherein R is selected from CHF₂, H, C₁₋₆ alkyl, CH₂F, CHCl₂ and CClF₂;
(ii) either a chiral (D)-cyclic amide base or (L)-cyclic amide base of formula I(x) 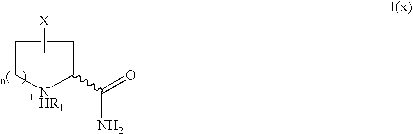
wherein
n is 0, 1 or 2;
R₁ is H or C₁₋₆ alkyl; and
X is H, halo or C₁₋₆ alkyl,
wherein the chiral base used is either (D) for separation of (R)-mandelic acids, or (L) for separation of (S)-mandelic acids; and optionally
(iii) an additional racemising organic amine base;
wherein the acid:total of cyclic amide base and optional organic amine base molar ratio is at least 1:1; provided that the cyclic amide base is present in a molar ratio of at least 0.75; and
wherein the mixture may optionally contain water in the range of 2% to 15% by volume of solvent;
(b) heating the mixture above ambient temperature and
(c) separating the respective (R)/(D) or (S)/(L) mandelic acid-cyclic amide salt of formula IIa;

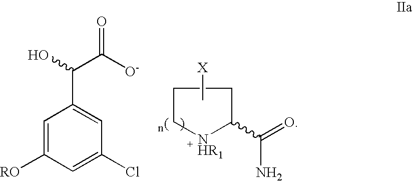

12. The process according to claim 1, wherein the (R)/(D) mandelic acid/cyclic amide salt is of formula VI;

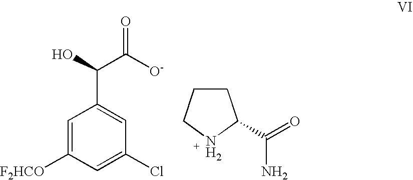

13. The process according to claim 1, wherein the solvent used is selected from ethyl acetate, iso-propyl acetate, n-butyl acetate, MIBK, DMF, DMSO, DMA, dioxane, N-methylpyrrolidinone, acetonitrile, acetone, 2-butanone, tert-butyl methyl ether, ethanol, 2-propanol, heptane, iso-octane and a mixture of any of these solvents.
